(19) United States
(12) Patent Application Publication
(10) Pub. No.: US 2020/0063204 A1
(43) Pub. Date: Feb. 27, 2020

(54) PERSONALIZED LCPUFA SUPPLEMENTATION TO EXPECTANT MOTHERS TO REDUCE RISK OF ASTHMA IN THEIR OFFSPRING

(71) Applicant: Den Selvejende Institution Dansk BorneAstma Center, Gentofte (DK)

(72) Inventors: Hans Bisgaard, Charlottenlund (DK); Klaus Bønnelykke, Allerød (DK)

(73) Assignee: Den Selvejende Institution Dansk BørneAstma Center, Gentofte (DK)

(21) Appl. No.: 16/465,768

(22) PCT Filed: Dec. 1, 2017
(86) PCT No.: PCT/EP2017/081154

§ 371 Date: May 31, 2019

(30) Foreign Application Priority Data
Dec. 2, 2016 (DK) PA 2016 00743

(51) Int. Cl. C12Q 1/6883; G01N 33/92; C12Q 1/6827

(52) U.S. Cl. CPC C12Q 1/6883; C12Q 2600/156; C12Q 1/6827; G01N 33/92

(57) ABSTRACT

A method for targeted (precision) prevention of asthma or other wheezing disorders or allergic rhinitis in children by restricting n-3 LCPUFA supplementation to expectant mothers where such supplementation has highest preventive effect.

### FIELD OF THE INVENTION

The present invention relates to a method of identifying expectant mothers who would benefit from supplementation with one or more n-3 long chain polyunsaturated fatty acids (LCPUFAs) in order to reduce the risk of asthma or other wheezing disorders or allergic rhinitis in their child. This is done by combined assessment of maternal blood levels of one or more specific n-3 LCPUFAs (eicosapentaenoic acid (20:5n-3, EPA), docosahexaenoic acid (22:6n-3, DHA) and/or docosapentaenoic acid (22:5n-3, DPA)), and maternal fatty acid desaturase (FADS) genotype. This method allows for targeted (precision) prevention of asthma or other wheezing disorders or allergic rhinitis in children by restricting n-3 LCPUFA supplementation to expectant mothers where such supplementation has highest preventive effect. The present invention also relates to a method for prophylactic treatment against asthma or other wheezing disorders or allergic rhinitis in the offspring, to a composition comprising one or more long chain polyunsaturated fatty acids for use in the method, and to a kit for use in the method.

### BACKGROUND OF THE INVENTION

Asthma and other wheezing disorders is one of the main causes for health care utilization in childhood with an increasing prevalence in westernized countries in recent decades (1). Concomitantly, the use of vegetable oils and grain feeding of livestock has resulted in an increase in the intake of n-6 polyunsaturated fatty acids (PUFA) and a decrease in n-3 PUFA, especially the long chain PUFA (LCPUFA) eicosapentaenoic acid (20:5n-3, EPA) and docosahexaenoic acid (22:6n-3, DHA) particularly found in fish (2) and this has been hypothesized to be a potential cause of childhood asthma and allergies. LCPUFAs influence immune regulation and some observational studies have reported an association between n-3 LCPUFA deprived diet during pregnancy and increased risk of asthma and related disorders in the offspring (3-5). Randomized controlled trial (RCT) of n-3 LCPUFA supplementation to pregnant women have generally been under-powered and shown ambiguous results (6-9).

It has also been hypothesized that prenatal vitamin D supplementation reduces risk of asthma/recurrent wheeze in early childhood. However, it was shown that contrary to expectation a reduced risk of asthma/recurrent wheeze was inversely correlated with maternal vitamin D level before supplementation. Thus the effect was strongest among women with high vitamin D level at study entry whereas no significant effect was observed among women with lower vitamin D level at study entry (25).

Approximately 20% of children develop asthma-like symptoms in early life, and there is an urgent need for a better understanding of disease mechanisms to establish prevention strategies. For disease prevention, in some embodiments, a medical professional may intervene before the onset of disease in order to reduce disease risk. There is consequently a need for a targeted, and in some embodiments prophylactic, treatment of children at risk for developing asthma or other wheezing disorders or allergic rhinitis. The present invention fulfils such a need.

### OBJECT OF THE INVENTION

It is an object of embodiments of the invention to be able to prevent childhood asthma and allergic rhinitis and particularly to individualize such prevention so that supplementation is only given to individuals whom it will benefit. The present invention fulfils such a need. By allowing identification of expectant mothers with highest benefit from n-3 LCPUFA supplementation, it enables targeted prevention (precision prevention) of asthma in the offspring. Such targeted prevention will be cost-effective for individuals and society.

### SUMMARY OF THE INVENTION

It has been found by the present inventors that n-3 LCPUFA supplementation in pregnancy protects the offspring against the development of asthma. Specifically, the preventive effect of supplementation was seen in children of mothers with specific blood-levels of n-3 LCPUFA prior to the intervention and specific FADS gene variants. A similar tendency was seen for protection against allergic rhinitis in the offspring.

Based on these results, a method is proposed enabling identification of expectant mothers with high benefit from n-3 LCPUFA supplementation based on combined n-3 LCPUFA blood-level measurement and FADS gene analysis.

The present invention is based on the discovery by the present inventors that n-3 long chain polyunsaturated fatty acid (LCPUFA) supplementation in pregnancy protects the offspring against the development of asthma, specifically in children of mothers with low blood-levels of n-3 LCPUFA and risk gene variants of fatty acid desaturase (FADS) genes.

Thus, the present invention provides a method of testing if an expectant mother will benefit from n-3 LCPUFA supplementation based on combined FADS-gene analysis and n-3 LCPUFA blood-level measurement. This will allow prevention of asthma or other wheezing disorders or allergic rhinitis in the offspring of mothers where such supplementation will have a protective effect. The method comprises the combination of two steps: 1) analyzing a blood sample from said expectant mother for detecting the presence of the single nucleotide polymorphism (SNPs) rs1535 in the FADS gene region (or another SNP correlated to this variant) in combination with 2) measuring the levels of the n-3 LCPUFAs EPA, DHA and/or DPA in maternal blood.

It has specifically been found that supplementation with n-3 LCPUFA in the second half of pregnancy, such as in the third pregnancy trimester significantly reduces the risk of persistent wheeze and asthma in their young children if mothers have low to middle EPA+DHA blood levels and FADS risk genotypes (GG/AG at rs1535). A similar effect is seen for allergic rhinitis. However, the present invention is not limited thereto, and it is anticipated that supplementation with n-3 LCPUFA may be beneficial for a woman from the time of planning to become pregnant to birth of the child, such as from gestation to birth of the child, such as from week 20 post gestation to birth of the child, such as during the third trimester of pregnancy in order to reduce the risk of asthma or other wheezing disorders or allergic rhinitis in their child.

So, in a first aspect the present invention relates to a method for identifying expectant mothers who would benefit from supplementation with n-3 long chain polyunsaturated fatty acid (LCPUFA) in order to reduce the risk of asthma or other wheezing disorders or allergic rhinitis in their child, comprising:
i) analyzing a biological sample from an expectant mother for blood levels of one or more of eicosapentaenoic acid (20:5n-3, EPA), docosahexaenoic acid (22:6n-3, DHA) and docosapentaenoic acid (22:5n-3, DPA), and
ii) analyzing a biological sample from said expectant mother for maternal fatty acid desaturase (FADS) genotype,
   wherein a low to medium level of EPA, DHA and/or DPA and a genotype of the expectant mother characterized by the presence of the FADS genotypes GG or AG at SNP rs1535, or alternatively one of the gene variants rs99780 (T), rs5792235 (deletion), rs174574 (A), rs3834458 (deletion), rs174568 (T), rs174567 (G), rs174576 (A), rs174566 (G), rs174577 (A), rs174564 (G), rs35473591 (T), rs174562 (G), rs174554 (G), rs174553 (G), rs174551 (C), rs174550 (C), rs174547 (C), rs174546 (T), rs174545 (G), rs174578 (A), rs174580 (G), rs174581 (A), rs174583 (T), rs102275 (C), rs174537 (T), rs174536 (C), rs174535 (C), rs174533 (A), rs174584 (A), rs102274 (C), rs11320420 (deletion), rs174538 (A), rs174541 (C), rs4246215 (T), rs174530 (G), rs174529 (C), rs174528 (C), rs174594 (A), rs174599 (C), rs174601 (T), rs174592 (G), rs5792239 (deletion), rs57668028 (deletion), rs108499 (T), rs97384 (C), rs28456 (G), rs174561 (C), rs174560 (C), rs174557 (G), rs174556 (T), rs174555 (C), rs174549 (A), rs174548 (G), rs174544 (A), rs174534 (G), rs174600 (C), rs174598 (A), rs143352979 (deletion), or rs174559 (A), indicate an expectant mother who would benefit from supplementation with n-3 long chain polyunsaturated fatty acid (LCPUFA) in order to reduce the risk of asthma or other wheezing disorders or allergic rhinitis in her child.

In a second aspect the present invention relates to a method for prophylactic treatment against asthma or other wheezing disorders or allergic rhinitis of a child, comprising:
i) Identifying expectant mothers who would benefit from supplementation with n-3 long chain polyunsaturated fatty acid (LCPUFA) in order to reduce the risk of asthma or other wheezing disorders or allergic rhinitis in their child by the above method, and
ii) administering a prophylactically effective amount of one or more n-3 long chain polyunsaturated fatty acids (LCPUFAs) to said expectant mother.

In a third aspect the present invention relates to a composition comprising one or more n-3 long chain polyunsaturated fatty acids (LCPUFAs) for use in the method according to the invention.

In a fourth aspect the present invention relates to a kit for use in the method according to the invention.

### LEGENDS TO THE FIGURE

FIG. 1 shows the effect of n-3 long chain PUFA supplementation in pregnancy on risk of persistent wheeze or asthma during the first 5 years of life in the offspring;

FIG. 2 illustrates the effect of n-3 long chain PUFA supplementation on persistent wheeze or asthma development stratified by blood EPA+DHA level in the mothers at the day of randomization (pregnancy week 24) and maternal FADS genotype (at single nucleotide polymorphism rs1535). The effect of the intervention is described in terms of hazards ratios (HR) of persistent wheeze or asthma development before 5 years of age in each stratum. Blood level strata: Low=lower tertile (<4.3%), Medium=middle tertile (4.3-5.3%), High=upper tertile (>5.3%). The dotted line delineates the 4 strata (grey boxes) with high expected benefit from supplementation (HR<0.6, meaning a relative risk reduction larger than 40%); and

FIG. 3 shows the effect of n-3 long chain PUFA supplementation on risk of persistent wheeze or asthma during the first 5 years of life stratified by mothers with medium to low blood EPA+DHA levels 5.3%) at the day of randomization and FADS risk genotype (AG/GG at rs1535) (left panel termed “High risk children”) vs. mothers with high EPA+DHA levels (>5.3%) and/or AA genotype (right panel termed “Low risk children”).

### DETAILED DISCLOSURE OF THE INVENTION

### Specific Embodiments of the Invention

By an expectant mother we mean a woman who is pregnant or plan to become pregnant.

The sequences of the FADS gene region are well known by one skilled in the art. The skilled man will immediately appreciate that the information presented herein relating to the human FADS gene region may easily be equated or correlated with a similar gene variant at a nearby location in relation to the FADS gene region. In particular, this will be relevant for highly correlated gene variants to a genotype characterized by the presence of the FADS genotypes GG or AG at SNP rs1535 (e.g. with a correlation higher than 0.7), including the following gene variants (the allele associated with high benefit from supplementation is marked in brackets): rs99780 (T), rs5792235 (deletion), rs174574 (A), rs3834458 (deletion), rs174568 (T), rs174567 (G), rs174576 (A), rs174566 (G), rs174577 (A), rs174564 (G), rs35473591 (T), rs174562 (G), rs174554 (G), rs174553 (G), rs174551 (C), rs174550 (C), rs174547 (C), rs174546 (T), rs174545 (G), rs174578 (A), rs174580 (G), rs174581 (A), rs174583 (T), rs102275 (C), rs174537 (T), rs174536 (C), rs174535 (C), rs174533 (A), rs174584 (A), rs102274 (C), rs11320420 (deletion), rs174538 (A), rs174541 (C), rs4246215 (T), rs174530 (G), rs174529 (C), rs174528 (C), rs174594 (A), rs174599 (C), rs174601 (T), rs174592 (G), rs5792239 (deletion), rs57668028 (deletion), rs108499 (T), rs97384 (C), rs28456 (G), rs174561 (C), rs174560 (C), rs174557 (G), rs174556 (T), rs174555 (C), rs174549 (A), rs174548 (G), rs174544 (A), rs174534 (G), rs174600 (C), rs174598 (A), rs143352979 (deletion), rs174559 (A).

In an embodiment of the invention step ii) of the method according to the invention comprises analyzing a biological sample from an expectant mother for a genotype characterized by the presence of the FADS genotypes GG or AG at SNP rs1535.

Typical techniques for detecting the mutation may include restriction fragment length polymorphism, hybridisation techniques, DNA sequencing, exonuclease resistance, microsequencing, solid phase extension using dideoxynucleotides (ddNTPs), extension in solution using ddNTPs, oligonucleotide ligation assays, methods for detecting single nucleotide polymorphisms such as dynamic allele-specific hybridisation, ligation chain reaction, mini-sequencing, DNA “chips”, allele-specific oligonucleotide hybridization with single or dual-labelled probes merged with polymerase chain reaction (PCR) or with molecular beacons, and others. Preferably, said technique for detecting a mutation is selected in the group comprising methods for detecting single nucleotide polymorphisms (SNPs) and short insertion/deletions.

In an embodiment of the invention step i) of the method according to the invention comprises analyzing a biological sample from an expectant mother for blood levels of EPA and/or DHA.

Assessment of EPA, DHA and/or DPA levels in maternal blood can be performed in whole blood, plasma, serum, or red blood cell membranes. Typical techniques are gas chromatography, red blood cell membrane composition and mass spectroscopy including liquid chromatography and tandem mass spectrometry (LC/MS/MS) measurement. EPA and DHA levels are preferably expressed as the sum of the two in relation to the mean level of total blood fatty acids.

In an embodiment of the invention the method for identifying expectant mothers who would benefit from supplementation with n-3 long chain polyunsaturated fatty acid (LCPUFA) in order to reduce the risk of asthma or other wheezing disorders or allergic rhinitis in their child, comprises a combination of:
i) analyzing a biological sample from an expectant mother for blood levels of eicosapentaenoic acid (20:5n-3, EPA) and/or docosahexaenoic acid (22:6n-3, DHA), and
ii) analyzing a biological sample from said expectant mother for maternal fatty acid desaturase (FADS) genotype,
   wherein a low to medium level of EPA and/or DHA and a genotype of the expectant mother characterized by the presence of the FADS genotypes GG or AG at the single nucleotide polymorphism rs1535 indicates an expectant mother who would benefit from supplementation with n-3 long chain polyunsaturated fatty acid (LCPUFA) in order to reduce the risk of asthma or other wheezing disorders or allergic rhinitis in her child.

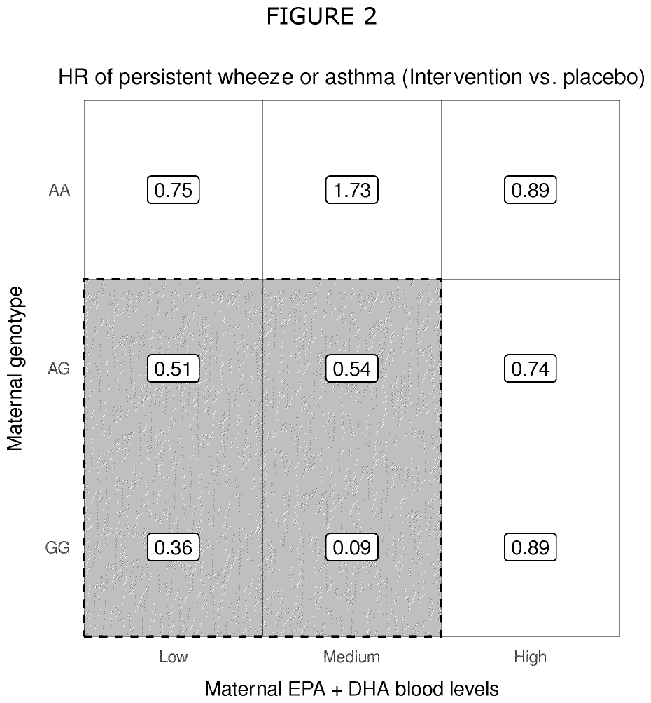

It has surprisingly been found that for identification of which expectant mothers will benefit from supplementation with n-3 LCPUFA in order to reduce the risk of asthma or other wheezing disorders or allergic rhinitis in her child there exists an interaction between the EPA, DHA, and/or DPA levels in a biological sample of an expectant mother and the FADS genotype. Thus a low or medium level of maternal EPA, DHA and/or DPA blood level is not sufficient to determine any benefit from n-3 LCPUFA supplementation, nor is determination of a specific FADS genotype (such as AG or GG at SNP rs1535), cf. FIG. 2. Only expectant women showing a combination of a low or medium level of maternal EPA, DHA and/or DPA and a specific genotype, such as AG or GG genotype at SNP rs1535 will benefit from n-3 LCPUFA supplementation.

In some embodiments a low to medium level of EPA, DHA and/or DPA refers to a level of EPA+DHA+DPA <=5.5% by weight (mean relative % of blood fatty acids) as measured by Gas Chromatography Mass Spectrometry (GC-MS) analysis of whole blood.

In some embodiments the n-3 long chain polyunsaturated fatty acids (LCPUFA's) are selected from the group consisting of eicosapentaenoic acid (20:5n-3, EPA), docosahexaenoic acid (22:6n-3, DHA), and docosapentaenoic acid (22:5n-3, DPA).

In some embodiments a supplement of one or more n-3 long chain polyunsaturated fatty acids (LCPUFA's) is administered in a daily dosage in the range of 1-5 g/day, such as 1-4 g/day, such as 2-3 g/day, such as 2-2.5 g/day, such as 2.4 g/day.

The kit for use in the method according to the invention comprises means for analyzing a biological sample for levels of one or more of eicosapentaenoic acid (20:5n-3, EPA), docosahexaenoic acid (22:6n-3, DHA), and/or docosapentaenoic acid (22:5n-3, DPA), and means for analyzing a biological sample for fatty acid desaturase (FADS) genotype. Means for analyzing a biological sample for levels of eicosapentaenoic acid (20:5n-3, EPA), docosahexaenoic acid (22:6n-3, DHA) and/or docosapentaenoic acid (22:5n-3, DPA) are well-known to a person skilled in the art. Non-limiting examples thereof comprise means for gas or liquid chromatography or mass spectroscopy. Means for analysing a biological sample for fatty acid desaturase (FADS) genotype are also well-known to a person skilled in the art. Non-limiting examples thereof comprise means for restriction fragment length polymorphism, hybridisation techniques, DNA sequencing, exonuclease resistance, microsequencing, solid phase extension using dideoxynucleotides (ddNTPs), extension in solution using ddNTPs, oligonucleotide ligation assays, methods for detecting single nucleotide polymorphisms such as dynamic allele-specific hybridisation, ligation chain reaction, mini-sequencing, DNA “chips”, allele-specific oligonucleotide hybridization with single or dual-labelled probes merged with polymerase chain reaction (PCR) or with molecular beacons.

In the following, the invention is described in more detail with reference to nucleic acid sequences and analysis of EPA and DHA blood levels. Yet, no limitation of the invention is intended by the details of the examples. Rather, the invention pertains to any embodiment, which comprises details which are not explicitly mentioned in the examples herein, but which the skilled person finds without undue effort.

### Example 1

### Methods

The study was approved by the Local Ethics Committee (H-B-2008-093) and the Danish Data Protection Agency (2008-41-2599) and registered at ClinicalTrials.gov (NCT00798226). Both parents gave written informed consent before enrolment.

A double-blinded RCT of n-3 LCPUFA supplementation during third trimester of pregnancy in an unselected group of 743 pregnant Danish women was conducted. This was the first well-powered RCT of n-3 LCPUFA supplementation with childhood asthma as primary endpoint. The children were prospectively monitored from birth with the primary aim to assess the risk of persistent wheeze/asthma during a 3 year double-blind follow-up period and an extended 2 years follow-up with extensive clinical phenotyping (10).

### Study Population

This was a single-center, double-blind, placebo-controlled, parallel-group study. Pregnant women were recruited at 22-26 weeks of pregnancy into the Copenhagen Prospective Studies on Asthma in Childhood₂₀₁₀ (COPSAC₂₀₁₀) pregnancy cohort (10), excluding women with >600 IU/day vitamin D intake and women having any endocrine, heart, or kidney disorders.

### Study Intervention

The women were randomized 1:1 at 24 weeks of pregnancy to daily supplementation of 2.4 g/day n-3 LCPUFA (55% EPA, and 37% DHA) in triacylglycerol form. Four 1 gram capsules (Incromega TG33/22, Croda Health Care, UK) were given in a double-blind manner with identically looking control supplementation capsules of olive oil (Pharmatech A/S, Norway) containing 72% n-9 oleic acid and 12% n-6 linoleic acid. EPA+DHA content and oxidation levels of PUFA and olive oil capsules were assured at two time-points during the study. The supplementation was continued until one week after delivery and the study intervention remained double-blinded until the youngest child turned 3 years. The follow-up continued observer-blind for the extended 2 years follow-up.

A subgroup of the children participating in this study (581) also participated in a nested, factorial designed, double-blind, RCT of 2400 IU/day vitamin D₃ supplementation to their mothers during third trimester of pregnancy (11).

### Adherence

Adherence to the n-3 LCPUFA intervention was assessed by comparing the number of returned capsules with the expectant.

### Maternal Blood Levels of EPA and DHA

EDTA blood samples were collected from the women in pregnancy week 24 (at the time of randomization) and at 1 week post partum, 0.01% 2,6-di-tert-butyl-4-methylphenol was added, and the samples were stored at −80° C. Fatty acid analyses, including EPA and DHA, were performed by Gas Chromatography Mass Spectrometry (GC-MS) analysis of whole blood as previously described (12,13).

### Maternal Fatty Acid Desaturase (FADS) Genotype

Maternal gene variation in FADS genes was tagged by genotyping of the single nucleotide polymorphism rs1535 (LGC Limited, Hoddesdon, UK) in mothers of European descent.

### Clinical Investigator-Diagnosed End-Points

COPSAC-pediatricians collected all information during clinical visits scheduled at 1 week, 1, 3, 6, 12, 18, 24, 30, and 36 months, and yearly thereafter. Additional acute visits were arranged whenever the children experienced lung symptoms. Asthma was diagnosed and treated by the COPSAC pediatricians following a predefined algorithm.

Daily diary cards were filled from birth by the parents monitoring troublesome lung symptoms including cough, wheeze, dyspnea severely affecting the well-being of the child. The diary cards were reviewed together with the parents by the COPSAC pediatricians, and double-checked after entering into the database.

Persistent wheeze and asthma (the primary end-point) was diagnosed based on a previously described quantitative symptom algorithm requiring (14,15): (1) verified diary recordings of 5 episodes of troublesome lung symptoms within 6 months, each lasting at least 3 consecutive days; (2) symptomatology typical of asthma including exercise-induced symptoms, prolonged nocturnal cough, and persistent cough outside common cold; (3) need for intermittent rescue use of inhaled B2-agonist; and (4) response to a 3-month course of inhaled corticosteroids and relapse upon ended treatment (14). Remission was defined by 12 months without relapse after cessation of inhaled corticosteroid treatment. The diagnosis was termed “persistent wheeze” until age 3 years and “asthma” hereafter.

Allergic rhinitis was diagnosed longitudinally by the COPSAC pediatricians from systematic interviews according to a predefined algorithm and defined as allergic rhinitis and/or conjunctivitis by age 5 years.

### Statistics

The effect of n-3 LCPUFA intervention on age at onset of persistent wheeze or asthma was assessed by Kaplan-Meier curves and quantified by Cox proportional hazards regression (P values correspond to Wald tests). Accordingly, the relative risk related to supplementation was assessed in terms of the Hazard Ratio (HR). Absolute disease risks were assessed from the Kaplan-Meier estimates. The dichotomized outcome of allergic rhinitis was analyzed using logistic regression. The data processing was conducted using statistical software R version 3.2.3, and CRAN packages survival and geepack (16).

Additional methodological details are outlined in the COPSAC₂₀₁₀ design paper (10).

### Results

### Baseline Characteristics

A total of 736 pregnant women were randomized to the intervention between November 2008 and November 2010, and the final child cohort consisted of 700 children including five pairs of twins. The baseline characteristics of the pregnant women and their children showed an unbiased randomization (p>0.1 in all comparisons).

### FADS Genotype

Maternal FADS genotype (rs1535) was obtained from mothers of 95% (N=660) of the children. Genotype call rate was 99.2% and there was no indication of deviation from Hardy-Weinberg equilibrium (p=0.35). Genotype distribution was AA: 284, AG: 291, GG:85. In line with previous studies, the minor allele (G) at rs1535 was associated with decreased maternal levels of EPA+DHA before the intervention (p=<0.0001), and increased levels of the “up-stream” substrates linoleic (18:2n-6) and α-linolenic acid (18:3n-3) (p<0.0001 and p=0.002, respectively).

### Adherence

71% (N=493) of the women had an intervention compliance >80% by capsule count with no differences between the n-3 LCPUFA and control group.

The n-3 LCPUFA supplementation resulted in increased blood levels of EPA+DHA (mean relative % of blood fatty acids) from 4.9% (SD 1.3) to 6.1% (SD 2.0) compared with a decrease from 4.9% (SD 1.2) to 3.8% (SD 1.1) in the control group. The mean difference in post-intervention levels between groups was 2.5% [2.2-2.7].

The clinical follow-up rate among children was 96% (N=664) by the end of the 3-year double blind period and 93% (N=647) by age 5 years. The average double blind follow-up age for the birth cohort was 4.2 years (SD 0.6) and the average follow-up age at the time of statistical analyses middle of 2016 was 6.2 years (SD 0.6).

### Persistent Wheeze/Asthma (Primary Outcome)

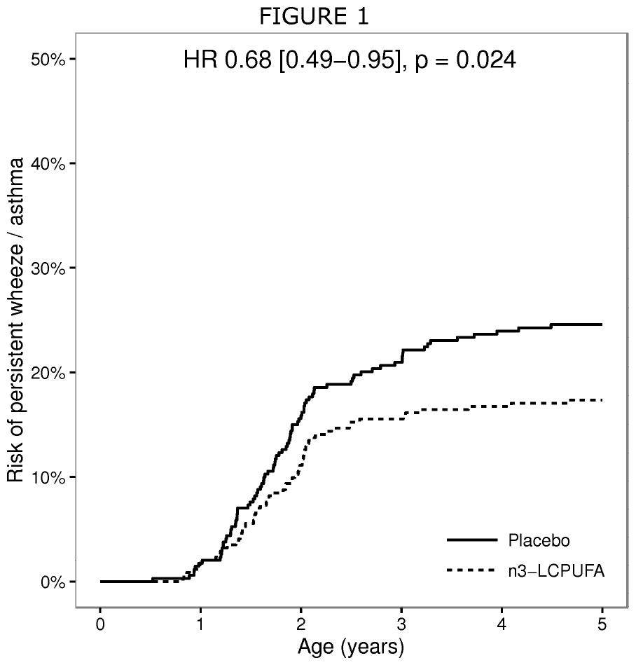

21.2% (N=137) of the children were diagnosed with persistent wheeze/asthma before age 5 years. The risk of persistent wheeze/asthma from birth to age 5 years was reduced by 32% in the n-3 LCPUFA supplemented group compared to the control group (HR 0.68 [0.49-0.95], p=0.02) (FIG. 1). The effect was similar during the initial double-blind clinical follow-up period from birth to age 3-5 years (16.9% vs. 23.7%, HR 0.69 [0.49-0.97], p=0.04), and at follow-up from birth to age 5-7 years (16.9% vs. 23.7%, HR 0.66 [0.47-0.91], p=0.01).

The preventive effect of n-3 LCPUFA supplementation was higher in children of women with low levels of EPA+DHA at randomization. The HRs of persistent wheeze/asthma in children of mothers in the lower (<4.3%), middle (4.3-5.3%) and upper (>5.3%) EPA+DHA tertiles were 0.47 [0.26-0.84], p=0.01; 0.76 [0.38-1.52], p=0.44; and 0.82 [0.44-1.50], p=0.51, respectively.

The preventive effect of n-3 LCPUFA was also modified by maternal FADS genotype with a stronger effect in offspring of women carrying the gene variant (G-allele at rs1535) associated with low EPA and DHA levels (GG: HR 0.37 [0.15-0.91], p=0.03), compared to children of mothers with AG genotype (HR 0.63 [0.37-1.09], p=0.10) and AA genotype (HR 0.91 [0.54-1.52], p=0.71).

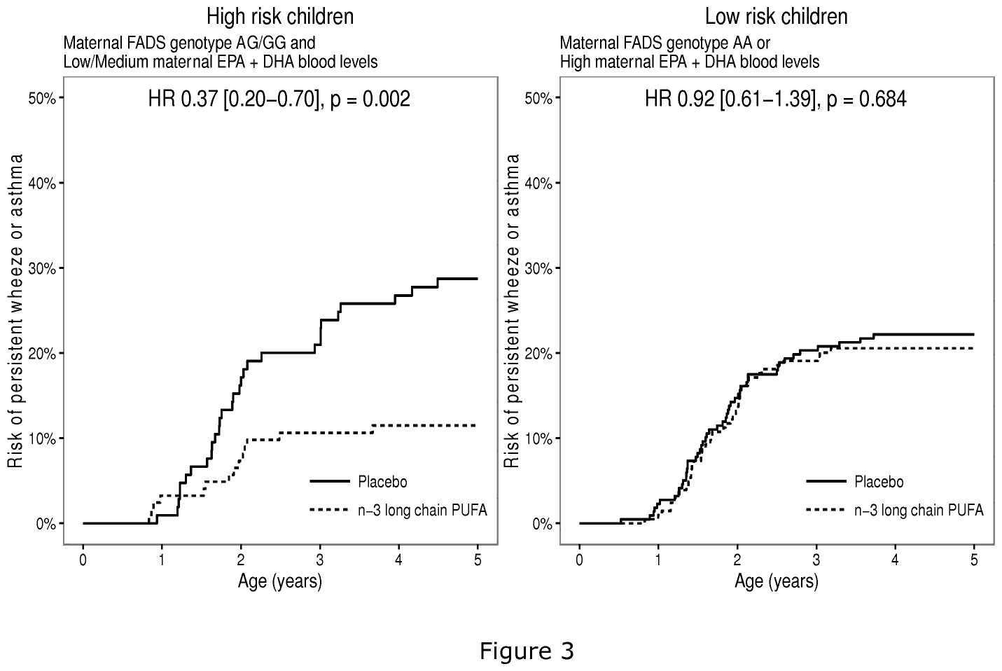

The interaction between maternal EPA+DHA blood levels and FADS genotype on effect of treatment is illustrated in FIG. 2. It is seen that the preventive effect of supplementation was highest (lowest HR) in children of mothers with low to medium EPA+DHA blood levels (<=5.3%) and at least one FADS risk allele (GG or AG genotype at rs1535). The relative risk reduction in all of these 4 strata (4 grey boxes) was more than 40% (HR<0.6). The protective effect of supplementation in the combined group of these 4 strata was more than 60% (HR 0.37 [0.20-0.70], p=0.002) (FIG. 3). This group of mothers and their children (“high risk children”) constituted 35% of the population.

In contrast there was a much smaller, or non-existent, protective effect of supplementation in the group of children of mothers with high EPA+DHA blood levels or non-risk (AA) genotype. The relative risk reduction in each all of these 5 strata was less than 30% (HR>0.7) (FIG. 2). It thus seemed that a high EPA+DHA blood level diminished the effect of supplementation in mothers with a risk genotype, and a non-risk genotype diminished the effect of supplementation in children with low EPA+DHA levels. The protective effect of supplementation in the combined group of these 5 strata was less than 10% and not statistically significant (HR 0.92 [0.61-1.39], p=0.68) (FIG. 3). This group of mothers and their children (“low-risk children”) constituted 65% of the population.

### Allergic Rhinitis (Secondary Outcome)

12% (78/645) of the children were diagnosed with allergic rhinitis by age 5 years. Supplementation with n-3 LCPUFA was not statistically significantly associated with allergic rhinitis (OR=0.70 [0.43-1.12], p=0.14). However, similarly to what was seen for asthma/persistent wheeze, the effect of supplementation was stronger in children of “high risk mothers” with low to medium EPA+DHA blood levels (<=5.3%) and FADS risk genotypes (OR=0.49 [0.20-1.17], p=0.14), compared the group of children of mothers with high EPA+DHA blood levels or non-risk (AA) genotype (OR=0.82 [0.45-1.52], p=0.53).

### Safety

The safety-profiles of n-3 LCPUFA and control supplementation appeared similar (Table 1).

### Discussion

### Main Results

The risk of persistent wheeze/asthma was reduced by approximately 30% in the first 5 years of life in children of women, who received daily supplementation of n-3 LCPUFA during third trimester of pregnancy. Specifically, this preventive effect was driven by children of women with low to medium pre-intervention EPA+DHA blood levels in combination with FADS gene risk variants. In this group of children, constituting 35% of the population, the protective effect was more than 60%. In the remaining children there was no significant effect of supplementation on asthma development. A similar tendency was seen for allergic rhinitis.

### Strengths and Limitation

The study is strengthened by the centralized longitudinal clinical follow-up with daily symptom recordings and regular as well as acute visits to our clinical research unit. COPSAC serves de facto as primary health care center solely for the birth cohort assuring a standardized approach to diagnosis and treatment. This greatly improves reliability of diagnoses over retrospective data collection based on reporting from the heterogeneous approach among community doctors, and this approach has previously provided strong statistical power despite comparatively low numbers in our mother-child cohorts (14,15,18,19).

We observed a higher effect of the intervention in children of mothers with low levels of EPA+DHA, and in children of mothers with FADS genotype associated with low EPA+DHA blood levels. This pattern is biologically plausible and thereby strongly supports that the observed effect from the intervention is a causative effect of n-3 LCPUFA supplementation.

For proof of concept, we used a high dose of n-3 LCPUFA supplement corresponding to an estimated 10-fold increase relative to the normal daily intake of n-3 LCPUFA in Denmark (20) and an estimated 20-fold increase relative to other countries, such as Canada and the US (21,22), while it is possible that a lower dose would suffice. The FADS SNP genotyped (rs1535) was chosen because it, and its proxies in close linkage disequilibrium, has been reported to be associated with n3-LCPUFA levels in a genome-wide association study (GWAS) (23), and rs1535 has been associated with blood levels of EPA and DHA during pregnancy (17).

### Interpretation

This is the first RCT on n-3 LCPUFA supplementation during pregnancy showing an effect on asthma in the offspring in a study designed and powered to explore such preventive effect. Our findings are in line with a previous, smaller, RCT on n-3 LCPUFA in pregnancy, initially conducted to study impact on pregnancy duration, where asthma in the offspring was later analyzed as a secondary outcome (9). Another RCT of n-3 LCPUFA supplementation in pregnancy followed 706 infants from birth to age 3 years (8). However, only an underpowered analysis on “atopic asthma” (asthma in combination with sensitization) was performed, showing no effect.

Importantly, we showed for the first time that expectant mothers benefitting most from supplementation can be identified by combined assessment of maternal EPA+DHA levels and FADS genotype, allowing precision prevention in mothers with low dietary intake and risk genotype. This will increase cost-effectiveness of this prevention and avert inconvenience and potential side effects, although yet not known, from unnecessary intake of supplements.

The maternal baseline intake of n-3 LCPUFA was high in our study population from a global perspective. The lower tertile of whole blood levels of EPA+DHA was <4.3% in our study compared to the reported average of 2.5-2.8% in North American populations (21,22). Similarly, our lower tertile of EPA+DHA intake was <315 mg/d compared to an estimated average intake in North American populations of 120 mg/d (21,22), and a global average intake of 163 mg/d with more than 80% of the global population consuming <250 mg/d (24). Our observation of a stronger intervention effect in children of mothers with lowest EPA+DHA levels and intakes therefore suggests that a sizeable effect may be expectant from supplement in other populations worldwide.

### CONCLUSION

Supplementation with n-3 LCPUFA in third pregnancy trimester significantly reduced the risk of persistent wheeze and asthma in their young children if mothers had low to middle EPA+DHA blood levels and FADS risk genotypes (AA/AG at rs1535). A similar effect was seen for allergic rhinitis.

These findings allow for a precision prevention strategy that can relieve the burden of asthmatic disorders in children.

TABLE 1
Safety assessment

| | Randomization % (N) | | | | |
|---|---|---|---|---|---|
| | n-3 long | | | | p- |
| | chain PUFA | | Control | | value |
| Adverse Events | 50% | (365) | 50% | (371) | — |
| Any maternal | | | | | |
| Death | 0% | (0) | 0% | (0) | — |
| Intrauterine death | 1% | (2) | 1% | (2) | 0.99 |
| Gestational diabetes | 2% | (6) | 3% | (10) | 0.32 |
| Preeclampsia | 5% | (17) | 4% | (15) | 0.69 |
| Days hospitalized after birth, mean | 2.9 | (2.7) | 2.8 | (2.8) | 0.51 |
| (SD) | | | | | |
| Mother hospitalized >5 days | 10% | (34) | 10% | (35) | 0.99 |
| Emergency caesarean section | 14% | (52) | 11% | (41) | 0.20 |
| Antibiotics in third pregnancy | 18% | (65) | 17% | (63) | 0.78 |
| trimester | | | | | |
| Infection in third pregnancy trimester | 28% | (96) | 33% | (118) | 0.10 |
| Any infant | | | | | |
| Death | 0% | (0) | 0% | (0) | — |
| Extremely preterm (<28 weeks) birth | 0% | (1) | 0% | (1) | 0.99 |
| Very preterm (28 to <32 weeks) birth | 1% | (2) | 1% | (3) | 0.66 |
| Moderate to late preterm (32 to <37 | 3% | (12) | 4% | (15) | 0.58 |
| weeks) birth | | | | | |
| Child hospitalized after birth | 12% | (40) | 11% | (39) | 0.88 |
| Any congenital malformation | 5% | (20) | 6% | (24) | 0.56 |

### LIST OF REFERENCES

1. Eder W, Ege M J, von Mutius E. The Asthma Epidemic. N Engl J Med. 2006 Nov. 23; 355:2226-35.
2. Leaf A, Weber P C. A new era for science in nutrition. Am J Clin Nutr. 1987 May; 45(5 Suppl):1048-53.
3. Blumer N, Renz H. Consumption of omega3-fatty acids during perinatal life: role in immuno-modulation and allergy prevention. J Perinat Med. 2007; 35 Suppl 1:S12-18.
4. Willers S M, Devereux G, Craig L C A, McNeill G, Wijga A H, Abou El-Magd W, et al. Maternal food consumption during pregnancy and asthma, respiratory and atopic symptoms in 5-year-old children. Thorax. 2007 September; 62(9):773-9.
5. Sausenthaler S, Koletzko S, Schaaf B, Lehmann I, Borte M, Herbarth O, et al. Maternal diet during pregnancy in relation to eczema and allergic sensitization in the offspring at 2 y of age. Am J Clin Nutr. 2007 Feb. 1; 85(2):530-7.
6. Dunstan J A, Mori T A, Barden A, Beilin L J, Taylor A L, Holt P G, et al. Fish oil supplementation in pregnancy modifies neonatal allergen-specific immune responses and clinical outcomes in infants at high risk of atopy: A randomized, controlled trial. J Allergy Clin Immunol. 2003 December; 112(6): 1178-84.
7. Furuhjelm C, Warstedt K, Larsson 3, Fredriksson M, Böttcher M F, Fälth-Magnusson K, et al. Fish oil supplementation in pregnancy and lactation may decrease the risk of infant allergy. Acta Pdiatrica. 2009 Sep. 1; 98(9):1461-7.
8. Palmer D J, Sullivan T, Gold M S, Prescott S L, Heddle R, Gibson R A, et al. Randomized controlled trial of fish oil supplementation in pregnancy on childhood allergies. Allergy. 2013 Nov. 1; 68(11):1370-6.
9. Hansen S, Strom M, Maslova E, Dahl R, Hoffmann H J, Rytter D, et al. Fish oil supplementation during pregnancy and allergic respiratory disease in the adult offspring. J Allergy Clin Immunol. 2016 Apr. 25;
10. Bisgaard H, Vissing N H, Carson C G, Bischoff A L, Følsgaard NV, Kreiner-Møller E, et al. Deep phenotyping of the unselected COPSAC2010 birth cohort study. Clin Exp Allergy J Br Soc Allergy Clin Immunol. 2013 December; 43(12):1384-94.
11. Chawes B L, Bønnelykke K, Stokholm J, Vissing N H, Bjarnadóttir E, Schoos A-M M, et al. Effect of Vitamin D3 Supplementation During Pregnancy on Risk of Persistent Wheeze in the Offspring: A Randomized Clinical Trial. JAMA. 2016 Jan. 26; 315(4):353-61.
12. Armstrong J M, Metherel A H, Stark K D. Direct microwave transesterification of fingertip prick blood samples for fatty acid determinations. Lipids. 2008 February; 43(2):187-96.
13. Metherel A H, Taha A Y, Izadi H, Stark K D. The application of ultrasound energy to increase lipid extraction throughput of solid matrix samples (flaxseed). Prostaglandins Leukot Essent Fatty Acids. 2009 December; 81(5-6):417-23.
14. Bisgaard H, Hermansen M N, Loland L, Halkjaer L B, Buchvald F. Intermittent inhaled corticosteroids in infants with episodic wheezing. N Engl J Med. 2006 May 11; 354(19):1998-2005.
15. Bisgaard H, Bønnelykke K, Sleiman P M A, Brasholt M, Chawes B, Kreiner-Møller E, et al. Chromosome 17q21 gene variants are associated with asthma and exacerbations but not atopy in early childhood. Am J Respir Crit Care Med. 2009 Feb. 1; 179(3):179-85.
16. R Core Team. R: A language and environment for statistical computing. R Foundation for Statistical Computing, Vienna, Austria. [Internet]. Vienna, Austria; 2015. Available from: http://www.R-project.org/

17. Steer C D, Hibbeln J R, Golding J, Davey Smith G. Polyunsaturated fatty acid levels in blood during pregnancy, at birth and at 7 years: their associations with two common FADS2 polymorphisms. Hum Mol Genet. 2012 Apr. 1; 21(7):1504-12.
18. Bisgaard H, Hermansen M N, Buchvald F, Loland L, Halkjaer L B, Bønnelykke K, et al. Childhood asthma after bacterial colonization of the airway in neonates. N Engl J Med. 2007 Oct. 11; 357(15):1487-95.
19. Chawes B L K, Buchvald F, Bischoff A L, Loland L, Hermansen M, Halkjaer L B, et al. Elevated exhaled nitric oxide in high-risk neonates precedes transient early but not persistent wheeze. Am J Respir Crit Care Med. 2010 Jul. 15; 182(2):138-42.
20. Olsen S F, Hansen H S, Sandstrom B, Jensen B. Erythrocyte levels compared with reported dietary intake of marine n-3 fatty acids in pregnant women. Br J Nutr. 1995 March; 73(3): 387-95.
21. Denomme J, Stark K D, Holub B J. Directly quantitated dietary (n-3) fatty acid intakes of pregnant Canadian women are lower than current dietary recommendations. J Nutr. 2005 February; 135(2):206-11.
22. Stark K D, Beblo S, Murthy M, Buda-Abela M, Janisse J, Rockett H, et al. Comparison of bloodstream fatty acid composition from African-American women at gestation, delivery, and postpartum. J Lipid Res. 2005 March; 46(3):516-25.
23. Lemaitre R N, Tanaka T, Tang W, Manichaikul A, Foy M, Kabagambe E K, et al. Genetic loci associated with plasma phospholipid n-3 fatty acids: a meta-analysis of genome-wide association studies from the CHARGE Consortium. PLoS Genet. 2011 July; 7(7):e1002193.
24. Micha R, Khatibzadeh S, Shi P, Fahimi S, Lim S, Andrews K G, et al. Global, regional, and national consumption levels of dietary fats and oils in 1990 and 2010: a systematic analysis including 266 country-specific nutrition surveys. BMJ. 2014; 348:g2272.
25. Wolsk, H M, Chawes, B L, Litonjua, A, Hollis, B W, Waage, J, Stokholm, Bønnelykke, K, Bisgaard, h, Weiss, S T, Prenatal vitamin D supplementation reduces risk of asthma/recurrent wheeze in early childhood: A combined analysis of two randomized controlled trials, PLOS ONE, https://doLorq/10.1371/Journal.pone.0186657, Oct. 27, 2017

1. A method for identifying expectant mothers who would benefit from supplementation with n-3 long chain polyunsaturated fatty acid (LCPUFA) in order to reduce the risk of asthma or other wheezing disorders or allergic rhinitis in their child, comprising:
i) analyzing a biological sample from an expectant mother for blood levels of one or more of eicosapentaenoic acid (20:5n-3, EPA), docosahexaenoic acid (22:6n-3, DHA), and/or docosapentaenoic acid (22:5n-3, DPA),
ii) analyzing a biological sample from said expectant mother for maternal fatty acid desaturase (FADS) genotype,
wherein a low to medium level of the sum of EPA, DHA and/or DPA and a genotype of the expectant mother characterized by the presence of the FADS genotypes GG or AG at single nucleotide polymorphism rs1535, or alternatively one of the gene variants rs99780 (T), rs5792235 (deletion), rs174574 (A), rs3834458 (deletion), rs174568 (T), rs174567 (G), rs174576 (A), rs174566 (G), rs174577 (A), rs174564 (G), rs35473591 (T), rs174562 (G), rs174554 (G), rs174553 (G), rs174551 (C), rs174550 (C), rs174547 (C), rs174546 (T), rs174545 (G), rs174578 (A), rs174580 (G), rs174581 (A), rs174583 (T), rs102275 (C), rs174537 (T), rs174536 (C), rs174535 (C), rs174533 (A), rs174584 (A), rs102274 (C), rs11320420 (deletion), rs174538 (A), rs174541 (C), rs4246215 (T), rs174530 (G), rs174529 (C), rs174528 (C), rs174594 (A), rs174599 (C), rs174601 (T), rs174592 (G), rs5792239 (deletion), rs57668028 (deletion), rs108499 (T), rs97384 (C), rs28456 (G), rs174561 (C), rs174560 (C), rs174557 (G), rs174556 (T), rs174555 (C), rs174549 (A), rs174548 (G), rs174544 (A), rs174534 (G), rs174600 (C), rs174598 (A), rs143352979 (deletion), rs174559 (A),
indicate an expectant mother who would benefit from supplementation with n-3 long chain polyunsaturated fatty acid (LCPUFA) in order to reduce the risk of asthma or other wheezing disorders in her child.

2. The method according to claim 1, wherein a low to medium level of EPA, DHA and/or DPA refers to a combined level of <=5.5% by weight (mean relative % of blood fatty acids) as measured by Gas Chromatography Mass Spectrometry (GC-MS) analysis of whole blood.

3. The method according to claim 1 or 2, wherein step i) comprises analyzing a biological sample from an expectant mother for blood levels of eicosapentaenoic acid (20:5n-3, EPA) and/or docosahexaenoic acid (22:6n-3, DHA).

4. The method according to any one of the preceding claims, wherein step ii) comprises analyzing a biological sample from an expectant mother for a genotype characterized by the presence of the FADS genotypes GG or AG at single nucleotide polymorphism rs1535.

5. A method for prophylactic treatment against asthma or other wheezing disorders or allergic rhinitis of a child, comprising:
i) Identifying expectant mothers who would benefit from supplementation with n-3 long chain polyunsaturated fatty acid (LCPUFA) in order to reduce the risk of asthma or other wheezing disorders or allergic rhinitis in their child by the method according to any one of claims 1-4, and
ii) administering a prophylactically effective amount of one or more n-3 long chain polyunsaturated fatty acids (LCPUFA's) to said expectant mother.

6. The method according to claim 5, wherein the n-3 long chain polyunsaturated fatty acids (LCPUFA's) are selected from the group consisting of eicosapentaenoic acid (20:5n-3, EPA), docosahexaenoic acid (22:6n-3, DHA), and docosapentaenoic acid (22:5n-3, DPA).

7. The method according to any one of claims 5-6, wherein administering one or more n-3 long chain polyunsaturated fatty acids (LCPUFA's) to said expectant mother is performed from the time of planning to become pregnant to birth of the child, such as from gestation to birth of the child, such as from week 20 post gestation to birth of the child, such as during the third trimester of pregnancy.

8. The method according to any one of claims 5-7, wherein a supplement of one or more n-3 long chain polyunsaturated fatty acids (LCPUFA's) is administered in a daily dosage in the range of 1-5 g/day, such as 1-4 g/day, such as 2-3 g/day, such as 2-2.5 g/day, such as 2.4 g/day.

9. The method according to any one of the preceding claims, wherein the biological sample is maternal blood selected from the group consisting of whole blood, plasma, serum, and red blood cell membranes.

10. A composition comprising one or more n-3 long chain polyunsaturated fatty acids (LCPUFA's) for use in the method according to any one of claims 5-9.

11. The composition according to claim 10, wherein the n-3 long chain polyunsaturated fatty acids (LCPUFA's) are selected from the group consisting of eicosapentaenoic acid (20:5n-3, EPA), docosahexaenoic acid (22:6n-3, DHA), and docosapentaenoic acid (22:5n-3, DPA).

12. A kit for use in the method according to any one of claims 1-9, comprising:
a. means for analyzing a biological sample for levels of one or more of eicosapentaenoic acid (20:5n-3, EPA), docosahexaenoic acid (22:6n-3, DHA), and docosapentaenoic acid (22:5n-3, DPA)
b. means for analyzing a biological sample for fatty acid desaturase (FADS) genotype.
